(19) United States
(12) Patent Application Publication
(10) Pub. No.: US 2006/0264970 A1
(43) Pub. Date: Nov. 23, 2006

(54) Phacoemulsification tip

(75) Inventors: Paul Ernest, Jackson, MI (US); Mikhail Boukhny, Laguna Niguel, CA (US); Eric Lee, Irvine, CA (US)

Correspondence Address: ALCON, IP LEGAL, TB4-8, 6201 SOUTH FREEWAY, FORT WORTH, TX, 76134, US

(73) Assignee: ALCON, INC.

(21) Appl. No.: 11/133,651

(22) Filed: May 19, 2005

(51) Int. Cl. A61F 9/00

(52) U.S. Cl. 606/107; 604/22; 604/272

(57) ABSTRACT

A phacoemulsification tip having a distal end that is longer in the vertical direction than it is in the horizontal direction. When vibrated ultrasonically, such a shape causes the distal end of the phacoemulsification tip to have a slicing or chopping action as opposed to a more traditional emulsification action.

### BACKGROUND OF THE INVENTION

This invention relates generally to the field of phacoemulsification and more particularly to torsional phacoemulsification cutting tips.

The human eye in its simplest terms functions to provide vision by transmitting light through a clear outer portion called the cornea, and focusing the image by way of the lens onto the retina. The quality of the focused image depends on many factors including the size and shape of the eye, and the transparency of the cornea and lens.

When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an IOL.

In the United States, the majority of cataractous lenses are removed by a surgical technique called phacoemulsification. During this procedure, a thin phacoemulsification cutting tip is inserted into the diseased lens and vibrated ultrasonically. The vibrating cutting tip liquifies or emulsifies the lens so that the lens may be aspirated out of the eye. The diseased lens, once removed, is replaced by an artificial lens.

A typical ultrasonic surgical device suitable for ophthalmic procedures consists of an ultrasonically driven handpiece, an attached cutting tip, and irrigating sleeve and an electronic control console. The handpiece assembly is attached to the control console by an electric cable and flexible tubings. Through the electric cable, the console varies the power level transmitted by the handpiece to the attached cutting tip and the flexible tubings supply irrigation fluid to and draw aspiration fluid from the eye through the handpiece assembly.

The operative part of the handpiece is a centrally located, hollow resonating bar or horn directly attached to a set of piezoelectric crystals. The crystals supply the required ultrasonic vibration needed to drive both the horn and the attached cutting tip during phacoemulsification and are controlled by the console. The crystal/horn assembly is suspended within the hollow body or shell of the handpiece by flexible mountings. The handpiece body terminates in a reduced diameter portion or nosecone at the body's distal end. The nosecone is externally threaded to accept the irrigation sleeve. Likewise, the horn bore is internally threaded at its distal end to receive the external threads of the cutting tip. The irrigation sleeve also has an internally threaded bore that is screwed onto the external threads of the nosecone. The cutting tip is adjusted so that the tip projects only a predetermined amount past the open end of the irrigating sleeve. Ultrasonic handpieces and cutting tips are more fully described in U.S. Pat. Nos. 3,589,363; 4,223,676; 4,246,902; 4,493,694; 4,515,583; 4,589,415; 4,609,368; 4,869,715; 4,922,902; 4,989,583; 5,154,694 and 5,359,996, the entire contents of which are incorporated herein by reference.

In use, the ends of the cutting tip and irrigating sleeve are inserted into a small incision of predetermined width in the cornea, sclera, or other location. The cutting tip is ultrasonically vibrated along its longitudinal axis within the irrigating sleeve by the crystal-driven ultrasonic horn, thereby emulsifying the selected tissue in situ. The hollow bore of the cutting tip communicates with the bore in the horn that in turn communicates with the aspiration line from the handpiece to the console. A reduced pressure or vacuum source in the console draws or aspirates the emulsified tissue from the eye through the open end of the cutting tip, the cutting tip and horn bores and the aspiration line and into a collection device. The aspiration of emulsified tissue is aided by a saline flushing solution or irrigant that is injected into the surgical site through the small annular gap between the inside surface of the irrigating sleeve and the cutting tip.

Many surgical techniques require that the lens be broken into sections or fragments prior to being emulsified. One method of breaking the lens apart requires that a separate instrument or chopper be inserted into the eye. The chopper is used to manually break apart the lens prior to legs fragment removal.

One prior art tip, disclosed in U.S. Pat. No. 4,515,583, (Sorich) discloses a phacoemulsification tip that is oval or elliptical along the entire length of the shaft. Such a construction is argued to better fit within the surgical incision and to provide a wider tip for making a wider furrow during lens sculpting. The benefits of these features are described as reducing surgical time and the chances of tissue burns within the tight incision. However, a tip having such a construction, with the tip wider in the horizontal direction than in the vertical direction, is not suitable as a substitute for a second chopper instrument.

Therefore, a need continues to exist for a phacoemulsification tip that can be used as a chopper and as an emulsifier.

### BRIEF SUMMARY OF THE INVENTION

The present invention improves upon the prior art by providing a phacoemulsification tip having a distal end that is longer in the vertical direction than it is in the horizontal direction. When vibrated ultrasonically, such a shape causes the distal end of the phacoemulsification tip to have a slicing or chopping action as opposed to a more traditional emulsification action.

Accordingly, one objective of the present invention is to provide a phacoemulsification cutting tip having a distal end that is longer in the vertical direction than it is in the horizontal direction.

Another objective of the present invention is to provide a phacoemulsification cutting tip having a distal end of the phacoemulsification tip has a slicing or chopping action as opposed to a more traditional emulsification action when vibrated ultrasonically.

These and other advantages and objectives of the present invention will become apparent from the detailed description and claims that follow.

### BRIEF DESCRIPTION OF THE DRAWINGS

FIG. 1 is an enlarged perspective view of the phacoemulsification tip of the present invention.

FIG. 2 is an enlarged perspective view of the phacoemulsification tip of the present invention.

### DETAILED DESCRIPTION OF THE INVENTION

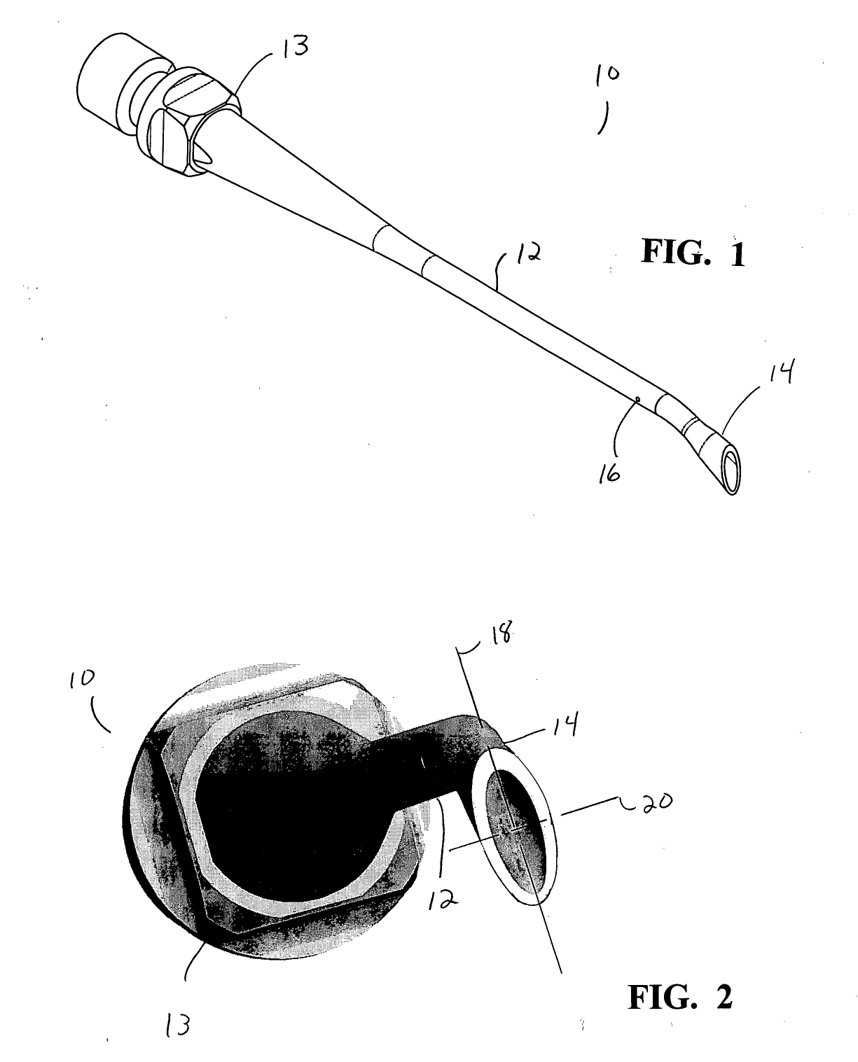

As best seen in FIG. 1, phacoemulsification tip 10 contains shaft 12 extending from hub 13. Shaft 12 is straight all the way to distal tip 14. Distal tip 14 may be angled or bent relative to the centerline of shaft 12. Shaft 12 may contain aspiration bypass hole 16.

As best seen in FIG. 2, distal tip 14 is elongated along vertical axis 18 relative to horizontal axis 20. By way of example, distal tip 14 may be sized between approximately 0.04 inches and 0.10 inches along vertical axis 18, with around 0.08 inches being preferred and between approximately 0.02 inches and 0.05 inches along horizontal axis 20, with around 0.03 inches being preferred.

Tip 10 is preferably made from stainless steel or titanium, but other materials may also be used. Tip 10 preferably has an overall length of between 0.50 inches and 1.50 inches, with 1.20 inches being most preferred. Tip 10 may be formed using conventional metalworking technology and preferably is electropolished to remove any burrs.

Shaft 12 is generally tubular, with an outside diameter of between 0.005 inches and 0.100 inches and an inside diameter of between 0.001 inches and 0.090 inches. Distal tip 14 may be cut square or cut at any suitable angle between 0° and 90°.

This description is given for purposes of illustration and explanation. It will be apparent to those skilled in the relevant art that changes and modifications may be made to the invention described above without departing from its scope or spirit.

I claim:

1. A phacoemulsification tip, comprising: a tubular shaft, the shaft having a distal tip, the distal tip being elongated along a vertical axis relative to a horizontal axis.

2. The phacoemulsification tip of claim 1 wherein the distal tip is angled relative the shaft.

3. The phacoemulsification tip of claim 1 wherein the shaft contains an aspiration bypass hole.
